(19) United States
(12) Patent Application Publication
(10) Pub. No.: US 2016/0058494 A1
(43) Pub. Date: Mar. 3, 2016

(54) MODULAR SURGICAL INSTRUMENT

(71) Applicant: Invuity, Inc., San Francisco, CA (US)

(72) Inventors: Alex Vayser, Mission Viejo, CA (US); Vladimir Zagatsky, San Francisco, CA (US); Fernando Erismann, New York, NY (US)

(21) Appl. No.: 14/821,516

(22) Filed: Aug. 7, 2015

Related U.S. Application Data
(60) Provisional application No. 62/043,071, filed on Aug. 28, 2014.

(51) Int. Cl. A61B 18/14; A61B 19/00

(52) U.S. Cl. CPC A61B 18/14; A61B 19/5212; A61B 2017/00424

(57) ABSTRACT

A modular surgical instrument includes an ergonomic handle, one or more service lines and a surgical tip. The handle is configured to be grasped by an operator, and includes a proximal portion, a distal portion, and a central portion. The one or more service lines are releasably coupled to the proximal portion of the handle, and the surgical tip is releasably coupled to the distal portion of the handle. The one or more service lines are operably coupled with the surgical tip. An optional control module may be disposed in a receptacle in the central portion of the handle and the control module may be adapted to control the operation of the surgical tip.

### CROSS-REFERENCE

The present application is a non-provisional of, and claims the benefit of U.S. Provisional Patent Application No. 62/043,071 (Attorney Docket No. 40556-736.101) filed Aug. 28, 2014; the entire contents of which are incorporated herein by reference.

### BACKGROUND OF THE INVENTION

### Field of the Invention

The present application generally relates to medical devices, systems and methods, and more particularly relates to modular hand held surgical instruments. These hand held devices are preferably easy to customize based on the anatomy being treated or the procedure being performed.

A number of surgical instruments include illumination for lighting the surgical field. These instruments may employ a fiber optic cable that is detachable from the main body of the surgical instrument. Also, the surgical instrument may also include a suction element which may require attachment of the surgical instrument to a vacuum line and/or a suction tube may be attached next to the fiber optic cable.

For example, during many spinal surgical procedures, it is common for surgeons to utilize multiple sizes of suction wands. If the suction tube has illumination, this requires the staff to remove the suction from the surgical field, detach both cables (fiber optic and suction tube) and attach a desired suction tip to the handle. The cost of each suction device is considerable when the illumination element is included with the entire product. Similarly, it may be desirable to replace one or more illumination elements from the surgical instrument during a procedure in order to provide more optimal lighting to the surgical field. This again may require a costly replacement of components. In another example, during electrosurgery, electrosurgical instruments and their corresponding cables may require replacement. Furthermore, in other surgical procedures, cables or surgical instruments may become contaminated if they fall out of the surgical field and thus replacement of the entire instrument can be costly and time consuming.

Therefore, it would be desirable to provide a surgical instrument handle that is easily coupled with or uncoupled from one or more working tips (e.g. suction tips and/or illumination tips) and that can be easily coupled and uncoupled to service lines (e.g. vacuum lines or fiber optic light input cables) required for the selected working tips.

Additionally, it would be desirable to provide a handle that can easily receive an appropriate control module for controlling the selected working tip. Such a control module is easily swapped out depending on the working tip selected.

Thus, a healthcare provider can maintain a supply of a variety of working tips such as an integrated suction tip with illumination element, or in some embodiments, several individual working tips may be used together such as a separate suction tip and a separate illumination tip. These tips may be easily swapped out as needed during surgery. The corresponding control module and service lines similarly can be easily be swapped out. At least some of these objectives will be satisfied by the embodiments described in this specification. Therefore, any working tip and corresponding service line may be easily coupled and uncoupled from the handle. If needed, any control module may also be easily coupled and uncoupled from the handle.

### SUMMARY OF THE INVENTION

The present invention generally relates to medical devices, systems and methods, and more particularly relates to a surgical instrument having a handle which is easily coupled and uncoupled from a number of working tips, and also easily coupled and uncoupled from the service lines required to operate the selected working tips. An optional control module may also be coupled or uncoupled to the handle to facilitate control of the working tip. In any of the embodiments, it may be preferable for one or more of the service lines or one or more of the working tips to be fixedly attached to the handle instead of being removably attached thereto.

In a first aspect, a modular surgical instrument comprises an ergonomic handle, one or more service lines and a surgical tip. The handle is configured to be grasped by an operator and has a proximal portion, a distal portion, and a central portion therebetween. The one or more service lines are releasably or fixedly coupled to the proximal portion of the handle and the surgical tip is releasably coupled to the distal portion of the handle. The one or more service lines are operably coupled with the surgical tip.

The one or more service lines may comprise a vacuum line configured to be coupled to a vacuum source so as to deliver suction to the surgical tip, a fiber optic cable configured to deliver light from an external source to an illumination element on the surgical tip, a power cable configured to deliver power from a power source to the working tip, or a data line configured to transmit data to or from another electronic component to the working tip. In some embodiments, multiple service lines may be combined into a single line. For example, a power and data line may be a single line that delivers power and transmits data to and from the working tip.

The surgical tip may comprise a suction tip, an illumination element, an electrosurgical tip with an electrode, a sensing element, an imaging element such as a camera, or combinations thereof. Other working tips such as electrical tips, cautery tips, neuromonitoring tips, image guided surgery tips, electromagnetic sensor tips, optical tracking tips, blood sensors, blood loss monitors, etc. may also be used. Tips with an illumination element alone or having an illumination element combined with another surgical working tip may use an optical waveguide as the illumination element. For example, when the working tip is a suction tip, an optical waveguide may be disposed around the suction tip. The surgical tip may also comprise a retractor for retracting tissue away from the incision. Such retractors may also be combined with any of the other working tips described elsewhere in this specification (e.g. illumination element, suction, electrosurgical tip, imaging, etc.)

The surgical tip may be threadably coupled to the distal portion of the handle or a bayonet connector may be used to releasably join the two components together. Other quick disconnect mechanisms known in the art may also be used, such as magnetic coupling.

The surgical instrument may further comprise a module that may be received in a hollow portion of a central portion of the handle and the module may be configured to be actuated or otherwise manipulated to facilitate control of the surgical tip. The module may comprise a vacuum control aperture that is configured to control suction strength when the surgical tip comprises a suction tip. The module may comprise a switch and actuation of the switch may control the illumination element or current delivery from the surgical tip to tissue when the surgical tip is an electrosurgical tip. In other embodiments the module may also include electronic circuits or processors that facilitate operation of the working tip (e.g. a camera control module for an imaging tip or electromagnetic tracking circuits for monitoring or controlling working tip position).

In another aspect, a modular surgical instrument comprises a handle, a surgical tip and a module. The handle has a proximal portion, a distal portion, and a hollow central portion therebetween. The handle is ergonomically shaped so that it may be easily grasped by an operator. The surgical tip is releasably or fixedly coupled to the distal portion of the handle, and the module is releasably disposed in the central portion of the handle. The module is configured to facilitate control of the surgical tip.

The surgical instrument may further comprise one or more service lines releasably coupled to the proximal portion of the handle. The one or more service lines may be operably coupled with the surgical tip and the module. The one or more surgical lines may comprise a vacuum line configured to deliver suction to the surgical tip, a fiber optic cable configured to deliver light to the surgical tip, a power cable configured to deliver power to the working tip, or a data line configured to transmit data to or from the working tip.

The module may comprise a vacuum control aperture configured to control suction strength when the surgical tip comprises a suction tip. The module may comprise a switch, and actuation of the switch may control illumination of the surgical field when the surgical tip comprises an illumination element. Actuation of the switch may control current delivery from the surgical tip to tissue in the surgical field when the surgical tip comprises an electrosurgical tip. In some embodiments, the module may include an integrated LED as an indicator light or for providing light to the surgical field. The LED may also be disposed on the working tip or in the handle.

The surgical tip may comprise a suction tip, an illumination element, an electrosurgical tip with an electrode, a sensing element, an imaging element, or combinations thereof. The illumination element may comprise an optical waveguide, and the surgical tip may comprise the optical waveguide disposed around the suction tip. The surgical tip may be threadably coupled to the distal portion of the handle, or the surgical tip may be coupled to the distal portion of the handle with a bayonet connector.

In another aspect, a method for performing a surgical procedure in a surgical field in a patient comprises providing an ergonomic handle, selecting a surgical tip, and releasably coupling the selected surgical tip. The handle has a proximal portion, a distal portion, and a central portion therebetween. The surgical tip is selected from a plurality of surgical tips, and the selected surgical tip is releasably or fixedly coupled to the distal portion of the handle. The method also comprises releasably coupling one or more service lines with the proximal portion of the handle. The service lines facilitate operation of the selected surgical tip. The method also comprises performing the surgical procedure on the patient with the selected surgical tip.

The selected surgical tip may comprise an illumination element and the surgical procedure may comprise illuminating the surgical field with light from the illumination element. The selected surgical tip may comprise a suction tip and the surgical tip may comprise suctioning the surgical field with a vacuum from the suction tip. In still other embodiments, the surgical tip may comprise an electrosurgical tip and the surgical procedure may comprise delivering current to tissue in the surgical field with the electrosurgical tip.

The method may further comprise detaching the selected surgical tip from the handle, selecting a second surgical tip from the plurality of surgical tips, releasably coupling the second surgical tip to the distal portion of the handle, and continuing performing the surgical procedure on the patient with the second surgical tip.

The central portion of the handle may be hollow and the method may comprise disposing a module in the hollow central portion of the handle, and actuating the module thereby controlling a function in the surgical tip. The module may comprise a vacuum control aperture, and the surgical tip may comprise a suction tip, and actuating the module may comprise adjusting obstruction of the vacuum control aperture thereby controlling suction strength delivered by the suction tip. The module may comprise a switch, and the surgical tip may comprise an illumination element, and actuating the module may comprise actuating the switch thereby controlling light delivered by the illumination element to the surgical field. The module may comprise a switch, and the surgical tip may comprise an electrosurgical tip having an electrode, and actuating the module may comprise actuating the switch thereby controlling current delivered by the electrosurgical tip to tissue in the surgical field.

Releasably coupling the one or more service lines may comprise releasably coupling a vacuum line, a fiber optic cable, a power cable, or a data line with the handle. Releasably coupling the selected surgical tip with the distal portion of the handle may comprise threadably coupling the selected surgical tip with the distal portion of the handle or bayonet coupling the selected surgical tip with the distal portion of the handle.

In another aspect, a method for performing a surgical procedure in a surgical field in a patient comprises providing an ergonomic handle having a proximal portion, a distal portion, a hollow central portion therebetween, and a surgical tip coupled to the distal portion of the handle. The method also comprises disposing a module in the hollow central portion of the handle, actuating the module thereby controlling a function in the surgical tip, and performing the surgical procedure on the patient with the surgical tip.

Optionally, the module may have an actuator mechanism for controlling actuation of a tip of the surgical tip. Exemplary actuation mechanisms may include motors, pull wires, or any other actuation mechanisms known in the art.

The method may further comprise selecting the surgical tip from a plurality of surgical tips and releasably coupling the selected surgical tip to the distal portion of the handle. Releasably coupling the selected surgical tip with the distal portion of the handle may comprise threadably coupling the selected surgical tip with the distal portion of the handle or bayonet coupling the selected surgical tip with the distal portion of the handle. Other coupling mechanisms are known in the art (e.g. magnetic coupling, slides, etc.) and they may also be used to couple the components together.

The method may further comprise releasably coupling one or more service lines with the proximal portion of the handle. Releasably coupling the one or more service lines may comprise releasably coupling a vacuum line, a fiber optic cable, a power cable, or a data line with the handle.

The surgical tip may comprise an illumination element and the surgical procedure may comprise illuminating the surgical field with light from the illumination element. The surgical tip may comprise a suction tip and the surgical procedure may comprise suctioning the surgical field with a vacuum from the suction tip. The surgical tip may comprise an electrosurgical tip and the surgical procedure may comprise delivering current to tissue in the surgical field with the electrosurgical tip.

The method may further comprise detaching the surgical tip from the handle, and selecting a second surgical tip from a plurality of surgical tips. The method may also comprise releasably coupling the second surgical tip to the distal portion of the handle, and continuing performing the surgical procedure on the patient with the second surgical tip.

The module may comprise a vacuum control aperture, and the surgical tip may comprise a suction tip. Actuating the module may comprise adjusting obstruction of the vacuum control aperture thereby controlling suction strength delivered by the suction tip. The module may comprise a switch, and the surgical tip may comprise an illumination element. Actuating the module may comprise actuating the switch thereby controlling light delivered by the illumination element to the surgical field. When the surgical tip is an electrosurgical tip having an electrode, actuating the switch may comprise controlling current delivered by the electrosurgical tip to tissue in the surgical field.

These and other embodiments are described in further detail in the following description related to the appended drawing figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic diagram of a modular surgical instrument.

FIG. 2 illustrates another schematic diagram of a modular surgical instrument.

FIG. 3 illustrates an exemplary working tip.

FIG. 4 illustrates another exemplary embodiment of a working tip.

FIG. 5 illustrates an exemplary handle.

FIG. 6 illustrates an exemplary handle with control module.

FIG. 7 illustrates an exemplary surgical suction instrument.

FIG. 8 illustrates an exemplary control module.

FIG. 9 illustrates an exemplary handle with service lines.

FIGS. 10A-10B illustrate an exemplary surgical suction instrument.

FIG. 11 illustrates another exemplary surgical suction instrument.

FIG. 12 highlights features of the embodiment in FIG. 11.

FIG. 13 illustrates an exemplary illuminated suction instrument.

FIG. 14 illustrates an exemplary surgical procedure.

FIG. 15 illustrates a tracking element on the surgical instrument.

### DETAILED DESCRIPTION OF THE INVENTION

Specific embodiments of the disclosed device, delivery system, and method will now be described with reference to the drawings. Nothing in this detailed description is intended to imply that any particular component, feature, or step is essential to the invention.

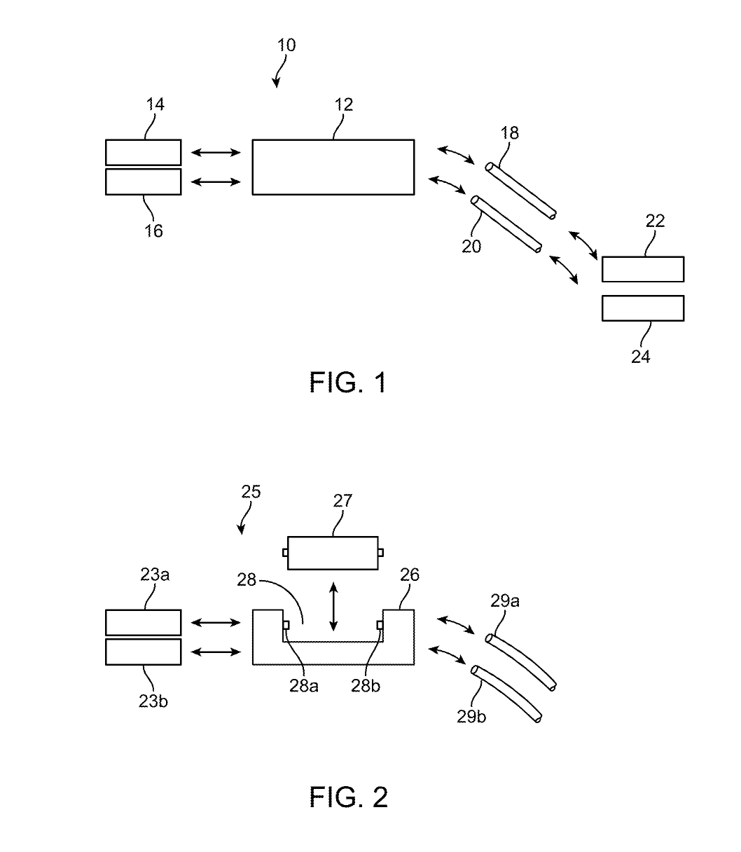

FIG. 1 illustrates a schematic diagram of an exemplary modular surgical instrument 10. In this embodiment, the surgical instrument 10 includes an ergonomically shaped handle 12, one or more working tips 14, 16 (two are illustrated for convenience) that couple to one or more positions on the distal portion of the handle (two positions are illustrated for convenience), and one or more service lines 18, 20 (two are illustrated for convenience) that couple to one or more proximal portions of the handle (two service lines are illustrated for convenience). A preferred embodiment has only a single working tip and only one or two service lines coupled to the handle. External sources 22, 24 may also be included. The one or more working tips 14, 16 may be any number of working tips such as a suction tip, an illuminated tip, an electrosurgical tip, a camera tip or other sensor tip, an electrode, etc. The working tips may have one type of tip and a second different type of tip coupled to the handle, or a working tip may have multiple functions combined into a single working tip which is coupled to the handle. For example, a suction tip and an illumination tip may be combined, or an illumination tip and a camera tip may be combined. One of skill in the art will appreciate that any combination of tips is possible. The working tips are preferably easily attachable and detachable from the distal portion of the handle. Any number of quick disconnects are known in the art such as bayonet connectors, threaded connectors, press fits, magnetic coupling, etc. and any of these may be used to couple and uncoupled the working tip from the handle. An electrical switch may also be disposed on the handle to electronically control the engagement and disengagement of the working tip from the handle. The handle optionally includes surface features such as texturing, knurling, finger grips, etc. to allow the user to comfortable grasp the handle.

The proximal portion of the handle similarly allows service lines 18, 20 to be easily coupled and uncoupled from the handle. Exemplary service lines include vacuum lines, power lines, fiber optic input lines, data lines, etc. The service line couples the working tip with the necessary source 22, 24. For example, when using a suction tip, the source is a vacuum, or when the working tip is an illumination tip, the source is a light source. For other working tips the source may be a power source such as electricity. For sensors, cameras or other working tips, the source may be source of power and a computer, processor, data storage device or any other electronic component required to operate the sensor, camera or other working tip. Optionally, the service line may be fixedly attached to the handle. For example, when the working tip includes an illumination element such as an optical waveguide, the service line may include a fiber optic input cable for optically coupling with an external light source. A portion of the fiber optic input cable may be fixedly coupled to the handle thereby creating a short pigtail section. Or, when the working tip is a suction tube, a short section of suction tubing may be fixedly coupled to the proximal end of the handle forming the pigtail. The free end of the service line that is fixedly coupled to the handle may also include a quick disconnect coupling that allows an extension service line to be coupled to the pigtail.

FIG. 2 illustrates another schematic diagram of an alternative exemplary embodiment of a modular surgical instrument 25 having a handle 26, one or more working tips 23a, 23b, one or more service lines 29a, 29b, and a control module 27. The working tips 23a, 23b generally take the same form as the working tips described in FIG. 1 and may be any of the working tips described in this specification. Similarly, the service lines 29a, 29b also generally take the same form as the service lines previously described in FIG. 1 and they may be any of the service lines described in this specification. The source or sources have been omitted from the schematic for clarity, although one of skill in the art will appreciate that one or more sources such as those previously described above or elsewhere in this specification may be used in this embodiment. The handle 26 includes a receptacle 28 for receiving the control module 27. Connections 28a and 28b allow the module to be operatively coupled with the working tip as well as the service lines. The module allows an operator to easily insert the corresponding control module into the handle receptacle when a working tip is selected. For example, in the case of a suction tip, the control module may have a suction control element for controlling suction strength. The suction control element may be an aperture that the operator may partially or fully cover with a finger to control suction strength. In the case of an electrosurgical tip, the control module may have a switch which allows the operator to control delivery of current to the patient from the electrosurgical tip. Thus, actuation of the switch in the control module allows an operator to control operation of the working tip. Other control modules similarly control operation of the corresponding working tip. Thus, for a camera the control module controls operation of the camera, for a sensor the control module controls operation of the sensor and transmission of data from the sensor to a source such as a data storage unit or a processor. Again, a control module may control multiple functions when the working tip performs multiple functions or when multiple working tips are coupled to the handle and used. In still other embodiments, the control module may include an actuation mechanism for controlling deflection of a distal portion of the working tip. For example, the control module may include motors or pull wires which control tip position. In still other embodiments, the control module may have other electronics disposed therein, such as an integrated sensor which measures blood count or other blood related diagnostic properties.

In any embodiment, preferably the working tip may be swapped out from the handle while leaving the service lines coupled to the handle. Or, in some embodiments where the service line is swapped out, preferably the service line may be swapped out while leaving the working tip coupled to the handle. The handle may accommodate any number of working tips such as suction tips, illumination tips, electrodes or other electrosurgical tips for cutting, coagulation, or other electrosurgery, a sensor tip for sensing a characteristic in the surgical field, a camera, or any other type of working tip. The working tips used may require electrical power, light, transmit data, or video signals, etc. thus the handle can accommodate any number of working tips and the necessary service line. Also, the appropriate control module may be easily coupled to the handle in order to control operation of the selected working tip.

Exemplary Working Tips

Any of the exemplary embodiments of working tips may be used in any of the modular surgical instruments described herein.

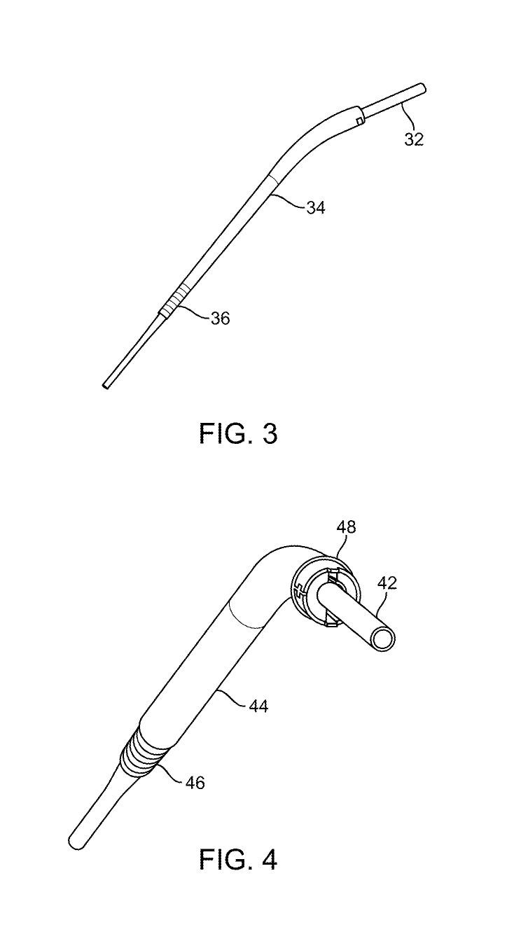

FIG. 3 illustrates an exemplary embodiment of a working tip which is a combined optical tip and suction tip. The tip includes a suction tube 32 and an illumination element 34. The working tip may be releasably coupled with the distal portion of the handle. The suction tip is used to suction fluids or other debris from the surgical field. An illumination element 34 is disposed over the suction tube and illuminates the surgical field while permitting simultaneous suctioning. Coatings or claddings may be disposed over the illumination element in order to enhance optical properties of the illumination element or light delivered therefrom. For example, if the illumination element is an optical waveguide, coatings or claddings such as fluorinated ethylene propylene (FEP) may be applied to the waveguide to enhance light transmission efficiency through the waveguide. Other coatings may be applied to obtain other desirable optical properties. Similarly, mechanical features such as standoffs may be used to maintain an air gap between the optical waveguide and the suction tube in order to help minimize light loss by contact between the two components. Optional surface features such as prisms or other optical surface features 36 allow light to be extracted and from the illumination element and directed to the surgical field. The illumination element may be an optical waveguide or any other illumination element such as a LED or incandescent bulb. In the case of an LED, the LED may be built into the working tip or into the handle. Thus, for example when the working tip is a suction tube, the LED or LEDs may be coupled to the suction tube to provide illumination, or the LED or LEDs may be coupled to a portion of the handle. The corresponding service lines would therefore preferably be a vacuum line and either a fiber optic cable for delivering light to the illumination element or a power cable for providing power to the illumination element such as an LED.

FIG. 4 illustrates an alternative embodiment of a working tip also having a suction tube 42, and illumination element 46 that generally takes the form of the illumination element in FIG. 3. A coating or cladding 44 such as those described previously (including an air gap) is disposed over the illumination element in order to minimize light loss due to contact with other materials. Additionally, a quick disconnect feature 48 such as threads or a bayonet mount may be used to attach and detach the working tip with the handle. Other quick disconnects such as press fits, detent mechanisms, etc. are known in the art and may also be used to couple the working tip with the handle.

Handle

Any of the embodiments of handles described herein may be used in any of the embodiments of surgical instruments described elsewhere in this specification.

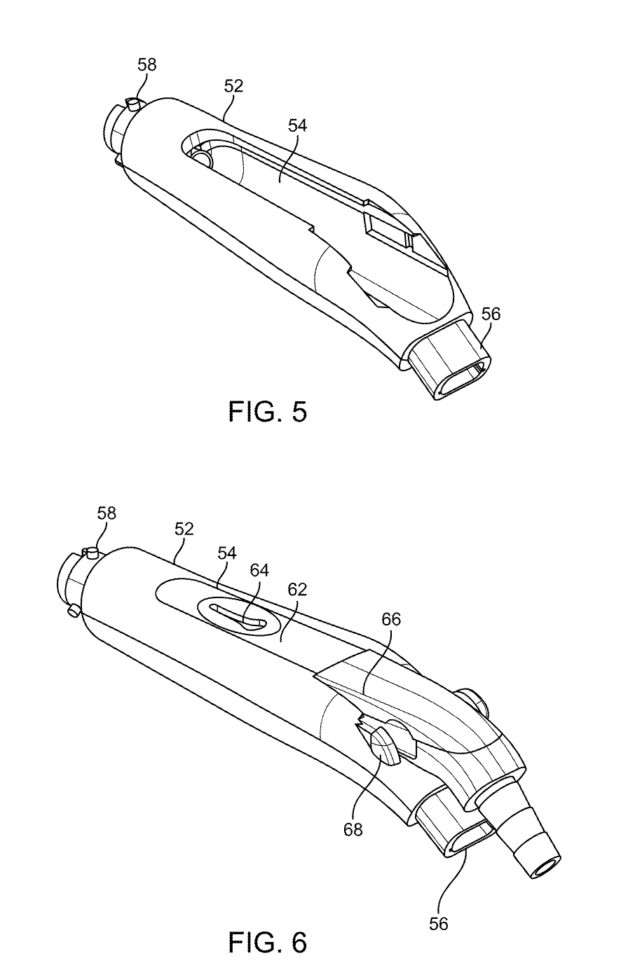

FIG. 5 illustrates an exemplary embodiment of a handle 52 having a proximal portion, a distal portion and a central or intermediate portion therebetween. The distal portion includes a quick disconnect feature 58 such as threads or a bayonet lock to allow a working tip (e.g. such as those described in this specification) to be easily attached and detached from the handle. This allows working tips to be easily swapped out during surgery depending on the procedure being performed and the anatomy being treated.

The proximal portion of the handle 56 allows the service lines to be coupled either directly or indirectly to the working tip. For example, fiber optic lines may run through the handle to the distal portion for coupling with an illumination element. Alternatively, the proximal portion may also include quick disconnects for coupling with service lines.

The intermediate portion of the handle optionally includes a receptacle 54 for receiving a control module. The control module is useful for controlling operation of the working tip. The control module may take the form of any control module described in this specification. Additional disclosure on the control module is disclosed below.

FIG. 6 illustrates the handle of FIG. 5 with a control module 62 disposed in the handle receptacle 54. Control module 62 in this embodiment has a slotted region 64 which is in fluid communication with the suction line and thus the operator may obstruct all or a portion of the slot 64 to thereby control suction strength at the working tip. Additionally, a quick release connector 66 is coupled to the proximal portion of the handle thereby allowing vacuum to be coupled with the module and the working tip. Here, quick release tabs 68 allow the vacuum connection to be easily attached and detached from the handle. Barbs on the quick release connection allow easy attachment to a vacuum line. In this embodiment, the control module is preferably integral with the service line connection.

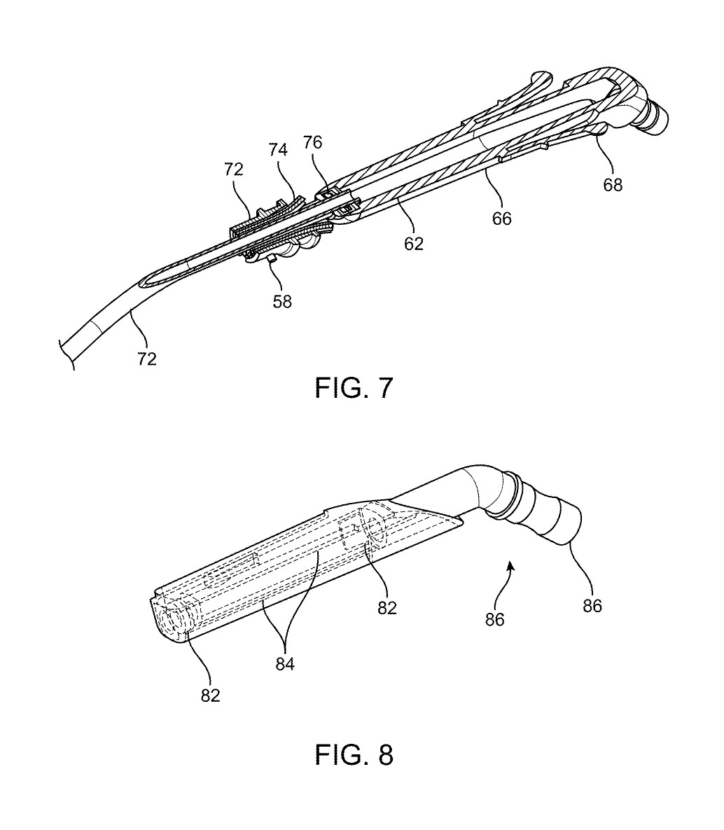

FIG. 7 illustrates a partial cross-section of the embodiment in FIG. 6 with an illuminated suction tip such as those described above coupled to the surgical instrument. Most of the handle has been removed from the figure for ease in viewing. The suction tube 72 is disposed in the quick disconnect connector 58 on the distal end of the handle and passes through an O-ring which creates a fluid seal against the suction tube. A channel in the control module 62 extends to the proximal end of the module to the barbed fitting allowing a vacuum line to be fluidly coupled with the suction tip. An illumination element (not illustrated) is disposed around the suction tube and fiber optics 74 in the distal handle portion 72 brings light or energy to illuminate the illumination element. The fiber optics may run through the central section of the handle toward the proximal portion of the handle, allowing a light input cable to be detachably coupled thereto.

FIG. 8 illustrates an exemplary embodiment of a control module 84 which may be disposed in the handle. The module 84 may include an O-ring adjacent a distal end to allow a fluid seal to be formed with the working tip. Additionally, a connector 86 may be coupled to the proximal end of the module to allow a service line to be easily attached and detached from the control module rather than with the handle.

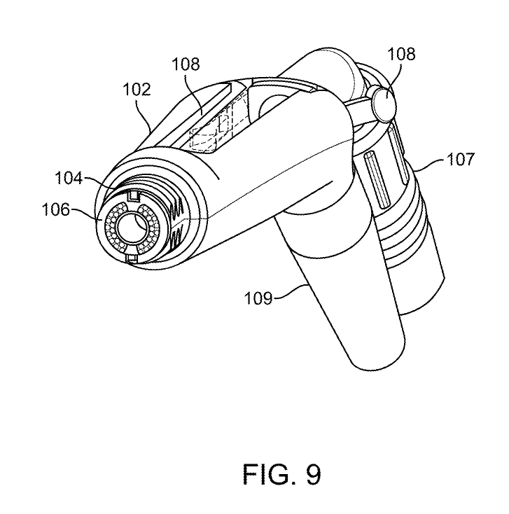

FIG. 9 illustrates a surgical instrument handle 102 with service lines coupled to the proximal end but no working tip attached to the handle. The distal portion of the handle has a threaded region 104 for attaching a working tip and also optical fibers 106 are disposed in the threaded region to pass light to an illumination element that may be coupled to the handle. The fiber bundle is split into two C-shaped arcs on either side of the distal portion of the handle, thereby helping to reduce profile of the device. The receptacle 108 in the intermediate section of the handle is sized to receive a control module. Service line 109 includes the fiber optics 106 that pass through the handle to the distal portion of the handle and service line 107 may be any service line such as power, data, fluid, suction, etc. In this embodiment the service line 107 is preferably a suction line. Quick release tabs 108 may be actuated to disconnect the service line from the handle. A threaded connector on service line 107 may also be used to allow the service line to be disconnected from the handle. As previously discussed, a service line may also be fixedly coupled to the handle forming a short pigtail section. In some embodiments, an illumination element such as an LED may be disposed in the handle. The illumination element may serve as an indicator light to provide operational status of the surgical instrument to an operator, or the illumination element may be used to provide light that illuminates the surgical field. In some embodiments, the handle may have both, an indicator or status light and an illumination element for provide light to the surgical field.

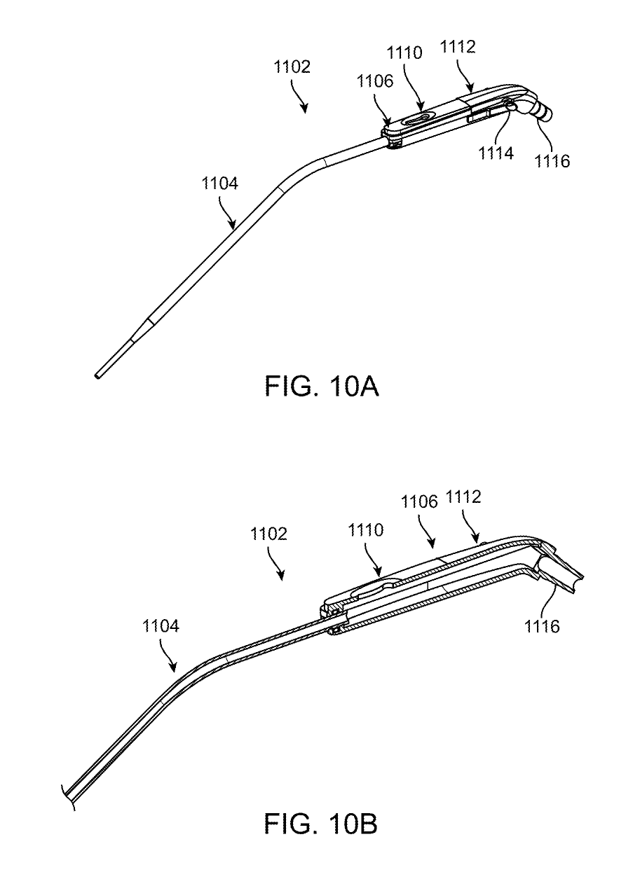

FIG. 10A illustrates an exemplary embodiment of a suction instrument 1102 having a suction tube 1104 as the releasable working tip and handle 1106. A control module 1112 is coupled to the handle and includes a suction control aperture 1110. Actuation of wings 1114 on the proximal quick release allow the control module to be easily engaged and disengaged with the handle. A barbed fitting 1116 allows a vacuum line to be easily coupled to the handle. Any number of geometries for the suction control aperture (also referred to as a vent) are possible. For example a larger hole when uncovered would substantially prevent suction from being applied to the distal suction tip. The operator may variable control the degree to which the hole is covered in order to vary the suction strength delivered to the distal suction tip. Exemplary embodiments of the vent may include circular holes, tapered slots, tear drop shapes as well as any other shape.

FIG. 10B illustrates a cross-section of the suction instrument in FIG. 10A and illustrates a suction path that extends from the suction tube tip through the handle and control module into the barbed fitting. An O-ring forms a fluid seal around the suction tube as it enters the handle. Other aspects of the surgical instrument are generally the same as other suction instruments described in this specification, including the handle, the control module with suction control and quick disconnect, and the barbed fitting for coupling with a vacuum line. In this or any embodiment, the control module may have a gross suction control as well as secondary, finer suction control. Either may include a valve or manual control such as an aperture that is covered by an operator's finger.

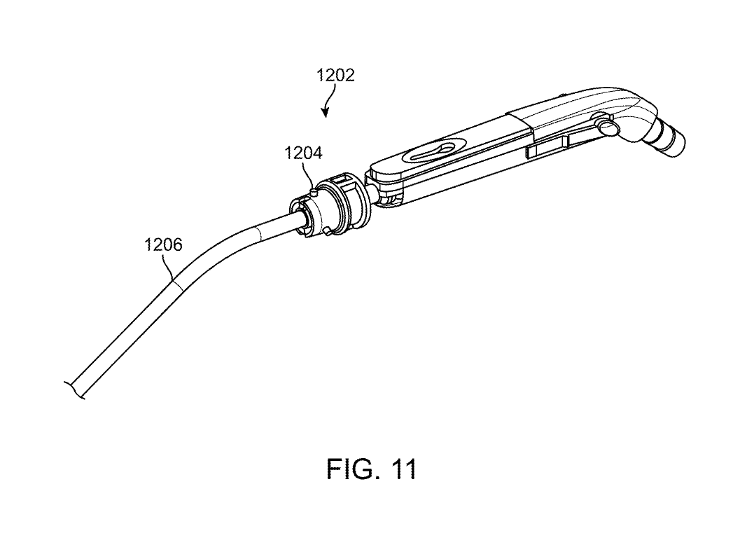

FIG. 11 illustrates a suction instrument 1202 that is similar to the embodiment in FIGS. 11A-11B with the major difference being that it also has a quick disconnect 1204 such as a bayonet connector which allows another tip to be coupled to the handle in addition the suction tube 1206. Optional fiber optics (not shown) may be disposed in the handle and pass through the quick disconnect 1204 in this embodiment.

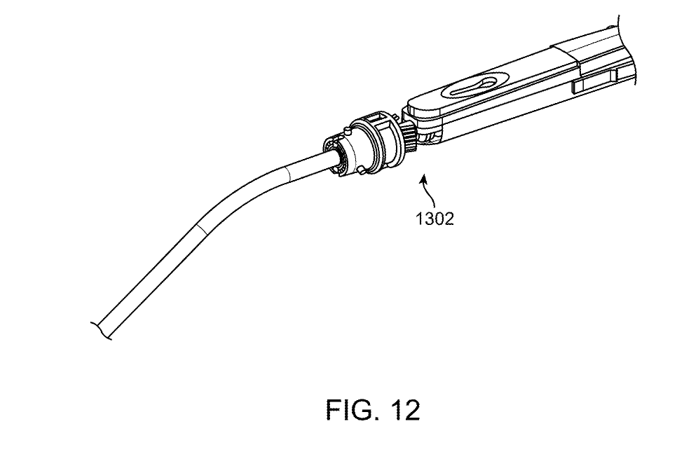

FIG. 12 illustrates the optional fiber optics 1302 passing through the quick disconnect. Other aspects of FIG. 12 are generally the same as those in FIG. 11.

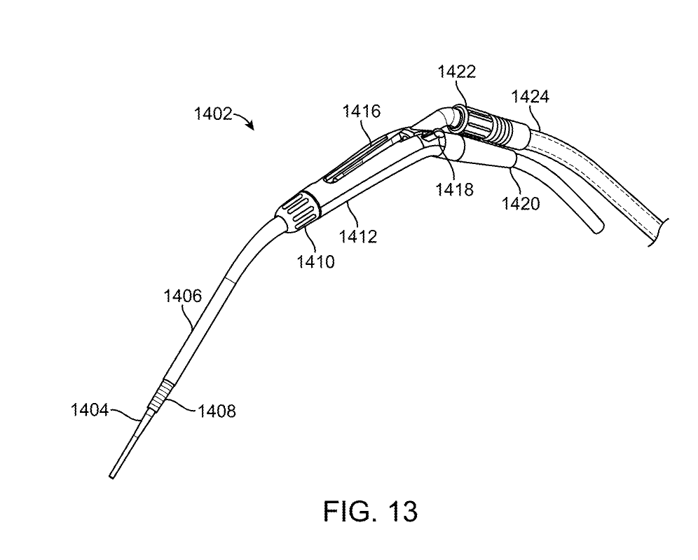

FIG. 13 illustrates an exemplary embodiment of a modular surgical instrument 1402 used for illumination and suction of a surgical field. The instrument 1402 includes a suction tube 1404, an optical illumination element 1408, cladding 1406, a quick disconnect 1410, a handle 1412, a control module 1416, quick disconnect 1418, vacuum line 1424, vacuum connector 1422, and fiber optic input cable 1420.

The suction tube 1404 allows blood or other debris to be evacuated from the surgical field. The illumination element 1408 is preferably an optical waveguide for delivering light from a light source to the surgical field. The illumination element may be tapered in a distal direction, and it may be disposed at least partially circumferentially, or completely circumferentially around the suction tube or other working tip. In some embodiments, the illumination element may have a concave or otherwise recessed region that receives the working tip in order to help reduce overall profile of the working tip. The illumination element, preferably an optical waveguide may also have surface features or other structures such as microstructures (e.g. prisms, lenslettes, or other structures) disposed on the tapered portion and/or the tip of the optical waveguide in order to help extract and direct light toward the surgical field. As disclosed elsewhere in this specification, any coatings or claddings may be disposed over all or selected portions of the illumination element in order to enhance the optical properties of the illumination element. For example, when the illumination element is an optical waveguide, FEP heat shrink may be disposed over a portion of the optical waveguide in order to prevent light leakage when there is contact between the waveguide and another object having a different index of refraction. In other embodiments, a layer of air or air gap disposed over the waveguide serves as the cladding.

Surface features such as prisms or other structures extract light from the waveguide and direct it to the surgical field. An outer coating or cladding such as air, fluorinated ethylene propylene (FEP) or any other material known in the art may be disposed over the waveguide to help prevent damage to the waveguide and also to prevent unwanted light loss therefrom. Threaded connector 1410 allows the suction tube 1404 to be easily attached and detached from the handle 1412. The handle also includes a control module 1416 disposed in the receptacle of the handle and includes a suction control aperture for controlling suction strength. Actuation of wings on quick disconnect 1418 allow the control module to be easily attached or detached from the handle. A fiber optic line 1420 is either releasably or fixedly coupled to the handle and a vacuum line 1424 is also releasably coupled to the handle with a threaded connector 1422.

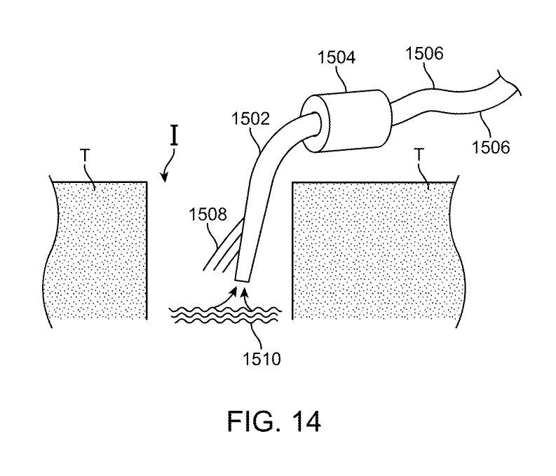

FIG. 14 illustrates an exemplary method of using an illuminated suction surgical instrument such as that in FIG. 13. The surgical instrument includes a suction tube 1502 that also includes an illumination element disposed around the suction tube, a handle 1504 and two service lines 1506 coupled to the proximal portion of the handle. The service lines 1506 in this example are a vacuum line and a fiber optic cable for bringing light from an external source to the illumination element. The instrument may be inserted into a surgical incision I form in tissue T in a patient. The suction may remove fluid or debris 1510 from the surgical field and light 1508 extracted from the illumination element, here preferably an optical waveguide illuminates the surgical field simultaneously during suctioning.

Malleability

Optionally, in any of the embodiments described herein, some portion of all of the working tip or tips may be formed from a malleable material such as silicone or any malleable material known in the art. Having all or a portion of the working tip allows a user to bend the tip into a desired configuration in order to conform with the local anatomy.

Image Guided Surgery

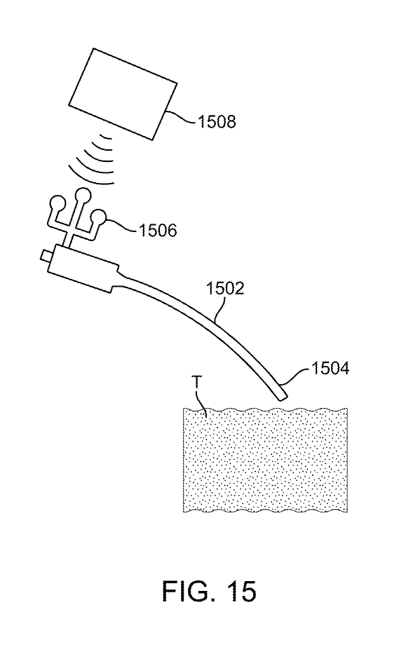

Optionally, in any of the embodiments described herein, the working tip may also be used for image guided surgery, as illustrated in FIG. 15. The working tip 1502 in this exemplary embodiment includes a suction tube with a tracking element 1506 coupled thereto. Here, the tracking element are fiducials which can be monitored using a camera or other detector and this allows the position of the working tip including its distal-most tip 1504 to be monitored or displayed on a monitor. This helps prevent unwanted damaged to adjacent tissue or anatomical structures. Optionally, the tracking element 1506 may be disposed on any portion of the device including the working tip, the handle, or on both the working tip and the handle. Tracking element 1506 may be disposed on any type of device or surgical instrument.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A modular surgical instrument, said surgical instrument comprising:
an ergonomic handle configured to be grasped by an operator, the handle having a proximal portion, a distal portion, and a central portion therebetween;
one or more service lines releasably coupled to the proximal portion of the handle; and
a surgical tip releasably coupled to the distal portion of the handle, wherein the one or more service lines are operably coupled with the surgical tip.

2. The surgical instrument of claim 1, wherein the one or more service lines comprise a vacuum line configured to deliver suction to the surgical tip, a fiber optic cable configured to deliver light to the surgical tip, a power cable configured to deliver power to the working tip, or a data line configured to transmit data to or from the working tip.

3. The surgical instrument of claim 1, wherein the surgical tip comprises a suction tip, an illumination element, an electrosurgical tip with an electrode, a sensing element, an imaging element, or combinations thereof.

4. The surgical instrument of claim 3, wherein the illumination element is an optical waveguide, and the surgical tip comprises the optical waveguide disposed around the suction tip.

5. The surgical instrument of claim 1, wherein the surgical tip is threadably coupled to the distal portion of the handle.

6. The surgical instrument of claim 1, wherein the surgical tip is coupled to the distal portion of the handle with a bayonet connector.

7. The surgical instrument of claim 1, further comprising a module, and wherein the central portion of the handle is hollow and sized to receive the module, wherein the module is configured to facilitate control of the surgical tip.

8. The surgical instrument of claim 7, wherein the module comprises a vacuum control aperture configured to control suction strength when the surgical tip comprises a suction tip.

9. The surgical instrument of claim 7, wherein the module comprises a switch, and wherein actuation of the switch controls illumination of the surgical field when the surgical tip comprises an illumination element, or wherein actuation of the switch controls current delivery from the surgical tip to tissue in the surgical field when the surgical tip comprises an electrosurgical tip.

10. A modular surgical instrument, said surgical instrument comprising:
an ergonomic handle configured to be grasped by an operator, the handle having a proximal portion, a distal portion, and a hollow central portion therebetween;
a surgical tip releasably coupled to the distal portion of the handle; and
a module releasably disposed in the central portion of the handle, wherein the module is configured to facilitate control of the surgical tip.

11. The surgical instrument of claim 10, further comprising one or more service lines releasably coupled to the proximal portion of the handle, and wherein the one or more service lines are operably coupled with the surgical tip and the module.

12. The surgical instrument of claim 11, wherein the one or more service lines comprise a vacuum line configured to deliver suction to the surgical tip, a fiber optic cable configured to deliver light to the surgical tip, a power cable configured to deliver power to the working tip, or a data line configured to transmit data to or from the working tip.

13. The surgical instrument of claim 10, wherein the module comprises a vacuum control aperture configured to control suction strength when the surgical tip comprises a suction tip.

14. The surgical instrument of claim 10, wherein the module comprises a switch, and wherein actuation of the switch controls illumination of the surgical field when the surgical tip comprises an illumination element, or wherein actuation of the switch controls current delivery from the surgical tip to tissue in the surgical field when the surgical tip comprises an electrosurgical tip.

15. The surgical instrument of claim 10, wherein the surgical tip comprises a suction tip, an illumination element, an electrosurgical tip with an electrode, a sensing element, an imaging element, or combinations thereof.

16. The surgical instrument of claim 15, wherein the illumination element is an optical waveguide, and the surgical tip comprises the optical waveguide disposed around the suction tip.

17. The surgical instrument of claim 10, wherein the surgical tip is threadably coupled to the distal portion of the handle.

18. The surgical instrument of claim 1, wherein the surgical tip is coupled to the distal portion of the handle with a bayonet connector.

19. A method for performing a surgical procedure in a surgical field in a patient, said method comprising:
providing an ergonomic handle, the handle having a proximal portion, a distal portion, and a central portion therebetween;
selecting a surgical tip from a plurality of surgical tips;
releasably coupling the selected surgical tip with the distal portion of the handle;
releasably coupling one or more service lines with the proximal portion of the handle, wherein the one or more service lines facilitate operation of the selected surgical tip; and
performing the surgical procedure on the patient with the selected surgical tip.

20. The method of claim 19, wherein the selected surgical tip comprises an illumination element and the surgical procedure comprises illuminating the surgical field with light from the illumination element.

21. The method of claim 19, wherein the selected surgical tip comprises a suction tip and the surgical procedure comprises suctioning the surgical field with a vacuum from the suction tip.

22. The method of claim 19, wherein the selected surgical tip comprises an electrosurgical tip and the surgical procedure comprises delivering current to tissue in the surgical field with the electrosurgical tip.

23. The method of claim 19, further comprising:
detaching the selected surgical tip from the handle;
selecting a second surgical tip from the plurality of surgical tips;
releasably coupling the second surgical tip to the distal portion of the handle; and
continuing performance the surgical procedure on the patient with the second surgical tip.

24. The method of claim 19, wherein the central portion of the handle is hollow, the method further comprising:
disposing a module in the hollow central portion of the handle; and
actuating the module thereby controlling a function in the surgical tip.

25. The method of claim 24, wherein the module comprises a vacuum control aperture, and the surgical tip comprises a suction tip, and wherein actuating the module comprises adjusting obstruction of the vacuum control aperture thereby controlling suction strength delivered by the suction tip.

26. The method of claim 24, wherein the module comprises a switch, and the surgical tip comprises an illumination element, and wherein actuating the module comprises actuating the switch thereby controlling light delivered by the illumination element to the surgical field.

27. The method of claim 24, wherein the module comprises a switch, and the surgical tip comprises an electrosurgical tip having an electrode, and wherein actuating the module comprises actuating the switch thereby controlling current delivered by the electrosurgical tip to tissue in the surgical field.

28. The method of claim 19, wherein releasably coupling one or more service lines comprises releasably coupling a vacuum line, a fiber optic cable, a power cable, or a data line with the handle.

29. The method of claim 19, wherein releasably coupling the selected surgical tip with the distal portion of the handle comprises threadably coupling the selected surgical tip with the distal portion of the handle or bayonet coupling the selected surgical tip with the distal portion of the handle.

30. A method for performing a surgical procedure in a surgical field in a patient, said method comprising:
providing an ergonomic handle having a proximal portion, a distal portion, a hollow central portion therebetween, and a surgical tip coupled to the distal portion of the handle;
disposing a module in the hollow central portion of the handle;
actuating the module thereby controlling a function in the surgical tip; and
performing the surgical procedure on the patient with the surgical tip.

31. The method of claim 30, further comprising selecting the surgical tip from a plurality of surgical tips and releasably coupling the selected surgical tip to the distal portion of the handle.

32. The method of claim 31, wherein releasably coupling the selected surgical tip with the distal portion of the handle comprises threadably coupling the selected surgical tip with the distal portion of the handle or bayonet coupling the selected surgical tip with the distal portion of the handle.

33. The method of claim 30, further comprising releasably coupling one or more service lines with the proximal portion of the handle.

34. The method of claim 33, wherein releasably coupling one or more service lines comprises releasably coupling a vacuum line, a fiber optic cable, a power cable, or a data line with the handle.

35. The method of claim 30, wherein the surgical tip comprises an illumination element and the surgical procedure comprises illuminating the surgical field with light from the illumination element.

36. The method of claim 30, wherein the surgical tip comprises a suction tip and the surgical procedure comprises suctioning the surgical field with a vacuum from the suction tip.

37. The method of claim 30, wherein the surgical tip comprises an electrosurgical tip and the surgical procedure comprises delivering current to tissue in the surgical field with the electrosurgical tip.

38. The method of claim 30, further comprising:
detaching the surgical tip from the handle;
selecting a second surgical tip from a plurality of surgical tips;
releasably coupling the second surgical tip to the distal portion of the handle; and
continuing performing the surgical procedure on the patient with the second surgical tip.

39. The method of claim 30, wherein the module comprises a vacuum control aperture, and the surgical tip comprises a suction tip, and wherein actuating the module comprises adjusting obstruction of the vacuum control aperture thereby controlling suction strength delivered by the suction tip.

40. The method of claim 30, wherein the module comprises a switch, and the surgical tip comprises an illumination element, and wherein actuating the module comprises actuating the switch thereby controlling light delivered by the illumination element to the surgical field.

41. The method of claim 24, wherein the module comprises a switch, and the surgical tip comprises an electrosurgical tip having an electrode, and wherein actuating the module comprises actuating the switch thereby controlling current delivered by the electrosurgical tip to tissue in the surgical field.
